(19) United States
(12) Patent Application Publication
(10) Pub. No.: US 2004/0186459 A1
(43) Pub. Date: Sep. 23, 2004

(54) Fluid delivery to cells and sensing properties of cells using nanotubes

(76) Inventors: Michael Shur, Latham, NY (US); Jonathan S. Dordick, Schenectady, NY (US); Pulickel M. Ajayan, Clifton Park, NY (US)

Correspondence Address: HESLIN ROTHENBERG FARLEY & MESITI PC, 5 COLUMBIA CIRCLE, ALBANY, NY, 12203, US

(21) Appl. No.: 10/393,437

(22) Filed: Mar. 20, 2003

(51) Int. Cl. A61M 31/00; C12N 15/00

(52) U.S. Cl. 604/500; 435/440

(57) ABSTRACT

A fluid delivery technique includes inserting a first end of a nanotube into the cell, connecting a second end of the nanotube to a fluid supply, and transferring fluid from the fluid supply into the cell via the nanotube. A technique for determining or sensing a property of a cell includes inserting two nanotubes into the cell, measuring at least one of a voltage and a resistance between the two nanotubes, and relating the at least one of the voltage and the resistance to a property of the cell. Other techniques and apparatus for fluid delivery to cell and sensing properties of a cell are also disclosed.

### FIELD OF THE INVENTION

The present invention relates generally to fluid delivery to cells and sensing properties of cells, and more particularly, to methods and systems for delivering fluid to cells using nanotubes and sensing properties of cells using nanotubes.

### BACKGROUND OF THE INVENTION

Conventional biosensing technologies are adept at identifying changes in the physiological state of cells in large populations (e.g., within fermentation or cell culture broths, or within various tissues). Recent developments in flow cytometry has enabled specific cellular organelles within individual cells to be probed, as well as optically-relevant macromolecules, such as green fluorescent protein-tagged molecules and structures. However, the rapid sensing of changes that occur to individual cells as a result of environmental or man-made perturbations remains a difficult problem. For example, it is important to elucidate the genetic changes that occur when cells are grown at different temperatures or pH's, or in the presence of various media components, toxic compounds, or carbon sources. Monitoring the phenotypic state of individual cells, as well as conditions at precise locations within the cell (e.g., cytoplasm, ER, nucleus, etc.) is critical to gain a more fundamental understanding of the cellular response.

There is a need for further methods and systems delivering fluid to cells using nanotubes and sensing properties of cells using nanotubes.

### SUMMARY OF THE INVENTION

In a first aspect, the present invention provides a method for delivering a fluid into a cell, the method includes inserting a first end of a nanotube into the cell, connecting a second end of the nanotube to a fluid supply, and transferring fluid from the fluid supply into the cell via the nanotube.

In a second aspect, the present invention provides a fluid delivery device for delivering a fluid to at least one cell. The fluid delivery device includes a support having a passageway therethrough and a nanotube disposed in the passageway. The nanotube includes a first end extending from a surface of the support, and a sidewall extends from the support to form a container for containing a fluid in fluid communication with a second end of the nanotube.

In a third aspect, the present invention provides a fluid delivery device for delivering a fluid to at least one cell. The fluid delivery device includes a support having a passageway therethrough which opens into a cutout on a surface of the support. At least one nanotube is disposed in the at least one passageway and a first end of the nanotube extends into the cutout. The cutout is configured for receiving at least one cell into which the first end of the nanotube is insertable.

In a fourth aspect, the present invention provides a method for forming a fluid delivery device in which the method includes providing a support having a passageway therein, forming a nanotube in the passageway, and removing a portion of the support to expose a first end of the nanotube.

In a fifth aspect, the present invention provides a method for fluidly connecting two or more cells in which the method includes inserting one end of a nanotube into a first cell, and inserting the other end of the nanotube into a second cell.

In a sixth aspect, the present invention provides a method for determining a property of a cell. The method includes inserting two nanotubes into the cell, measuring at least one of a voltage and a resistance between the two nanotubes, and relating the at least one of the voltage and the resistance to a property of the cell.

In a seventh aspect, the present invention provides a method for sensing a property of a cell. The method includes inserting two nanotubes into the cell, applying a voltage to the two nanotubes, and sensing a property of the cell.

In an eighth aspect, the present invention provides a method for determining a property of a cell. The method includes supporting the cell on a support, inserting a nanotube into the cell, measuring at least one of a voltage and a resistance between the support and the nanotube, and relating the at least one of the voltage and the resistance to a property of the cell.

In a ninth aspect, the present invention provides a method for sensing a property of a cell. The method includes supporting the cell on a support, inserting a nanotube into the cell, applying a voltage to the nanotube and the support, and sensing a property of the cell.

In a tenth aspect, the present invention provides an apparatus for use in measuring an impedance spectra of a cell. The apparatus includes a nonconductive support, two spaced-apart conductive pads, a first and second plurality of nanotubes each of which extends from a respective one of the two conductive pads, and wherein at least one of the first and the second plurality of nanotubes are spaced-apart a distance for receiving a cell therein and the first and second plurality of nanotubes define cavities for receiving individual cells therein.

In an eleventh aspect, the present invention provides a method for measuring the impedance spectra of at least one cell. The method includes providing a nonconductive support, depositing two spaced-apart conductive pads on the support, forming a first and a second plurality of nanotubes extending from the conductive pads wherein at least one of the first and the second plurality of nanotubes are spaced-apart a distance for receiving a cell therein and the first and the second plurality of nanotubes define a plurality of cavities for receiving individual cells therein, and measuring the impedance spectra using an impedance spectrometer attached to the conductive pads.

In a twelfth aspect, the present invention provides an apparatus for measuring an impedance spectra of at least one cell. The apparatus includes a nonconductive support having at least one passageway for receiving the at least one cell, and a pair of contacts disposed on opposite ends of the support and connectable to an impedance spectrometer.

In a thirteenth aspect, the present invention provides an apparatus for measuring a radiation spectra of at least one cell. The apparatus includes a support having at least one passageway for receiving the at least one cell, a radiation source disposed adjacent the support, and a detector disposed adjacent the support for detecting radiation from the at least one cell.

In a fourteenth aspect, the present invention provides a method of delivering a fluid and sensing a property of a cell. The method includes inserting a nanotube into the cell, introducing a fluid through the nanotube into the cell, detecting a property of the cell using the nanotube.

### BRIEF DESCRIPTION OF THE DRAWINGS

The subject matter which is regarded as the invention is particularly pointed out and distinctly claimed in the concluding portion of the specification. The invention, however, may best be understood by reference to the following detailed description of various embodiments and the accompanying drawings in which:

FIG. 1 is a perspective view of a cell pierced by a nanotube in accordance with the present invention;

FIG. 2 is a perspective view of a cell pierced by a nanotube bundle in accordance with the present invention;

FIG. 3 is a perspective view of a cell attached to a contact and pierced by a nanotube in accordance with the present invention;

FIG. 4 is a diagrammatic perspective view of the attachment of the cell to the contact of FIG. 3;

FIG. 5 is a perspective view of a nanotube fluid delivery device in accordance with the present invention;

FIG. 6 is a perspective view of the support of FIG. 5 having a pattern of passageways therein;

FIG. 7 is cross-sectional view of another fluid delivery device in accordance with the present invention;

FIG. 8 is a plan view of two cells connected by a nanotube in accordance with the present invention;

FIG. 9 is a plan view of a plurality of cells connected by nanotubes in accordance with the present invention;

FIG. 10 is a perspective view of a cell pierced by two spaced-apart nanotubes in accordance with the present invention;

FIGS. 11-13 are perspective views of the steps for forming an individual cell impedance spectra sensing apparatus in accordance with the present invention;

FIG. 14 is a view taken along line 14-14 of FIG. 13;

FIGS. 15-17 are perspective views of the steps for forming another individual cell impedance spectra sensing apparatus in accordance with the present invention;

FIG. 18 is a perspective view of a test fixture for cell impedance spectroscopy in accordance with the present invention;

FIGS. 19-22 are partial top views of the support of FIG. 18 with one or more cells contained within a passageway; and

FIG. 23 is a perspective view of a test fixture for absorption or photoluminescence spectroscopy.

### DETAILED DESCRIPTION OF THE INVENTION

The present invention is generally directed to the technique of using nanotubes which are inserted, e.g., partially inserted, into a cell for delivering fluid into the cell and for sensing properties of the cell. The technique also allows studying the physiological and phenotypic state of individual cells in their native environments. For example, the present invention may be practiced with small bacteria (e.g., diameters of about 1 micron to about 3 microns), yeasts and fungi (e.g., having a diameter of about 5 microns to about 15 microns), and mammalian cells (e.g., having a diameter of greater that about 30 microns).

Various aspects of the present invention employ nanotubes, such as carbon nanotubes, for probing cells at specific locations within the cell. The rigidity of nanotubes, as well as their hollow core, enables them to be used to transfer small molecules, proteins, or nucleic acids (e.g., DNA and RNA) into bacterial, fungal, or mammalian cells and into precise locations within these cells. The response of the cells to the introduction of these compounds can be determined through highly sensitive electrical measurements also using nanotubes.

Quasi-one-dimensional carbon whiskers or carbon nanotubes may be straight tubules with diameters in nanometers and properties close to that of an ideal graphite fiber. The uniqueness of the nanotube arises from its structure and the inherent “helicity” in the arrangement of hexagonal arrays on its surface honeycomb lattice. The helicity (local symmetry), along with its diameter (which determines the size of the repeating structural unit) introduces significant changes in the electronic density of states and hence provides a unique electronic character for the nanotubes. This novel electronic structure makes nanotubes either metallic or semiconducting. The combination of size, structure and topology results in nanotubes with important mechanical (e.g. high stability, strength and stiffness combined with low density, and elastic deformability), electrical, thermal, and surface properties (selectivity, surface chemistry). The structure of nanotubes remains distinctly different from traditional carbon fibers that have been industrially used for several decades.

Two varieties of nanotubes exist, which differ in the arrangement of graphene cylinders. Multi-wall nanotubes are collections of several concentric (co-axial) graphene cylinders and are larger (about 2 nanometers to about 30 nanometers in diameter) structures compared to single-wall nanotubes which are individual cylinders of about 1 nanometer to about 2 nanometers in diameter. Single-wall nanotubes show a strong tendency to bundle up into ropes consisting of aggregates of several tens of individual tubes organized into a one-dimensional lattice. Both types of nanotubes may be grown to microns of length. In recent years, work has focused on developing chemical vapor deposition techniques using catalyst particles and hydrocarbon precursors to grow nanotubes. As used herein the term “nanotube” is meant to include single-wall nanotubes, multi-wall nanotubes, bundles of nanotubes, and combinations thereof.

As described in greater detail, the fluid (e.g., gas or liquid) delivery devices and sensing devices may include one or more carbon nanotubes piercing one or more cells. The nanotubes may be arranged in an ordered array. The carbon nanotubes may be partially or completely incorporated into a non-organic material array comprising a periodic non-organic material array fabricated from, e.g., anodized aluminum. One or several nanotubes piercing the cell may also be used as electrodes for sensing the electrical properties and/or state of the cell and/or the chemical properties of the cell.

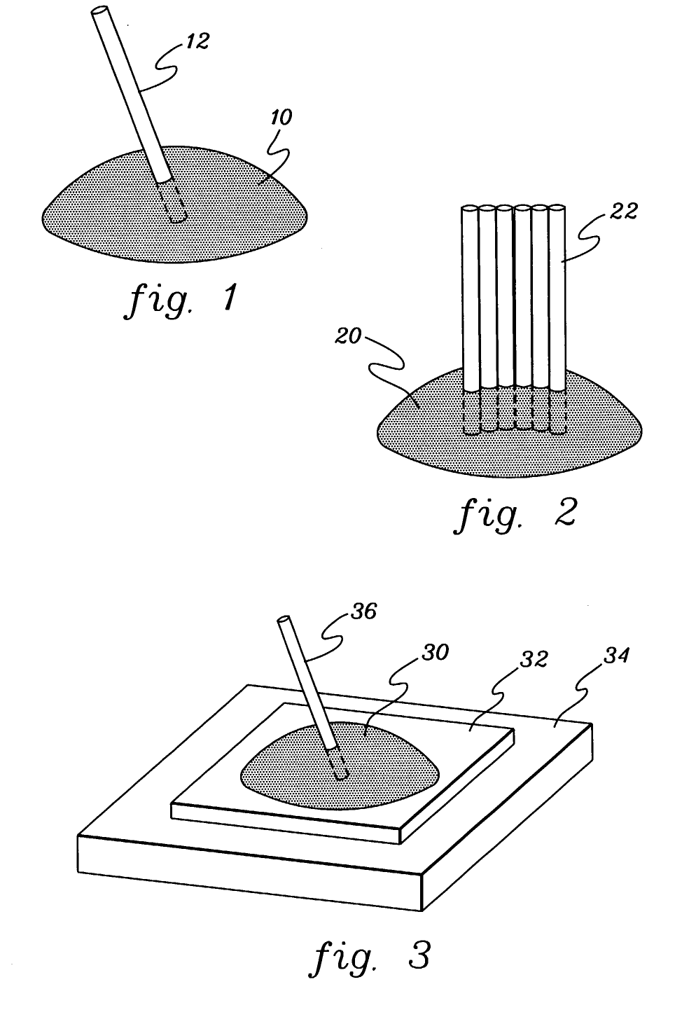

With reference now to the drawings, FIG. 1 illustrates a cell 10 pierced by a nanotube 12 in accordance with the present invention so that a portion of the nanotube is disposed in the cell. FIG. 2 illustrates a cell 20 pierced by a nanotube bundle 22 in accordance with the present invention so that a portion of the nanotube bundle is disposed in the cell. The arrangement shown in FIGS. 1 and 2 allow delivery of fluid such as a liquid or a gas into the cell.

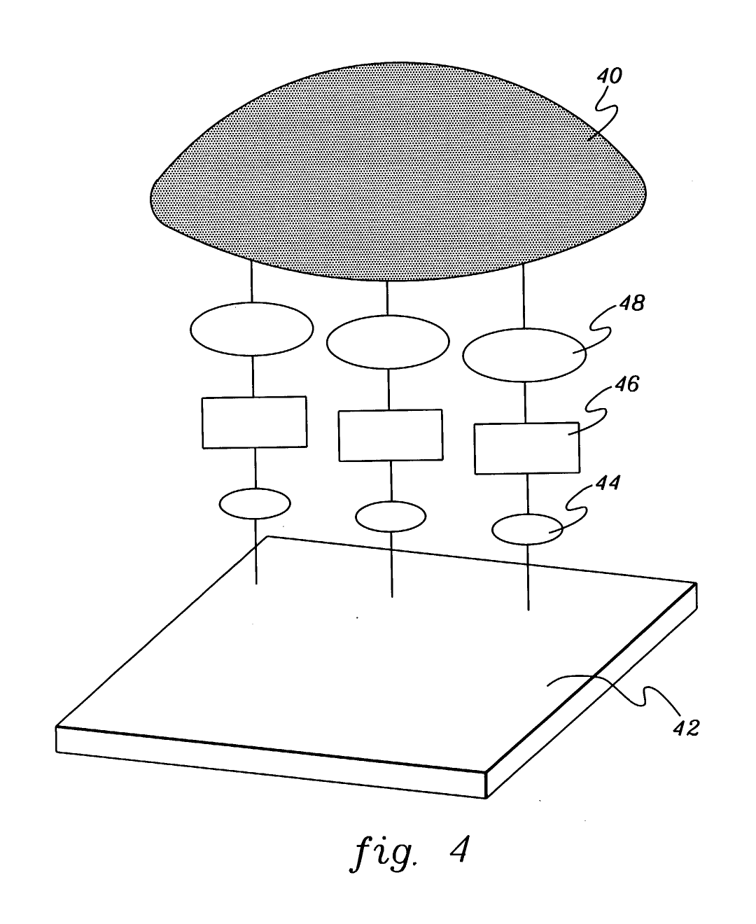

As illustrated in FIG. 3, a cell 30 may be attached to a patterned contact or support 32 (e.g., gold or other suitable material) which support is supported on a substrate 34, and cell 30 may be pierced by a nanotube 36. As best shown in FIG. 4, a cell 40 may be attached to a gold substrate 42 using biotin 44, stretovidin 46, and lectin 48 molecular chains. The length of the molecular chains may be about 20 nanometers to about 30 nanometers. Such chains are examples of attaching cells to a metal. This technique allows developing testing patterns for the cell to be pierced by the nanotubes.

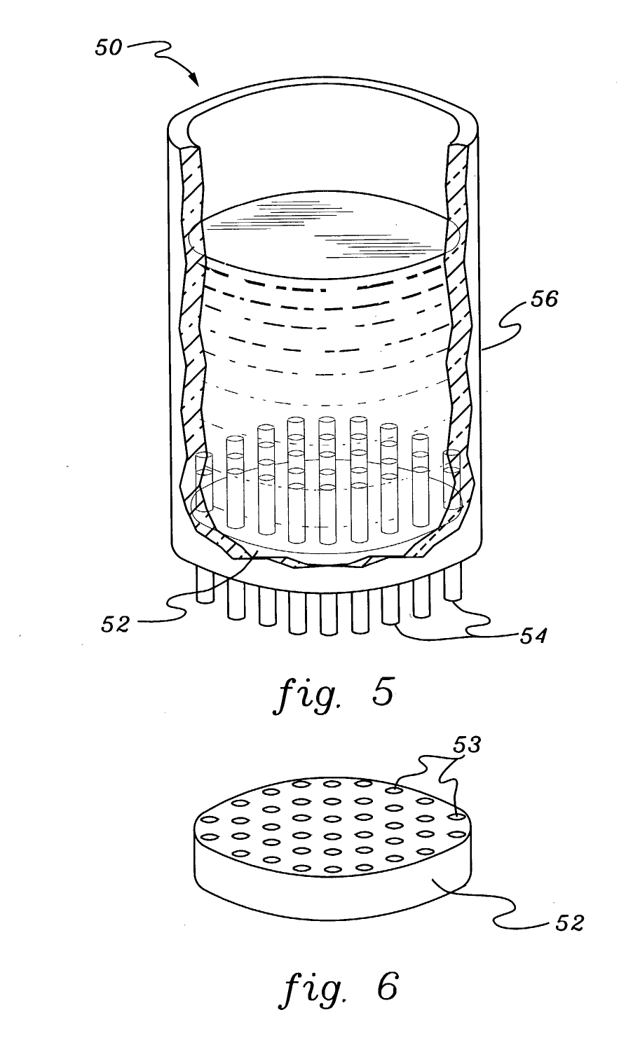

FIG. 5 illustrates a nanotube fluid cell delivery device 50 having an alumina template or support 52 with a plurality of nanotubes 54 extending therefrom. Support 52 may be attached to a container 56 for containing fluid therein. As shown in FIG. 6, alumina template or support 52 may be formed with a pattern or array of holes or passageways 53 therein. For example, separated individual nanotubes may be grow by using an electrochemically prepared porous support such as alumina and exposing the support to hydrocarbon vapor at high temperature thereby depositing nanotubes in the passageways of the support. The support can then be etched to expose either part or all of the nanotubes which can be effectively used as nanotube probe arrays as shown in FIG. 5.

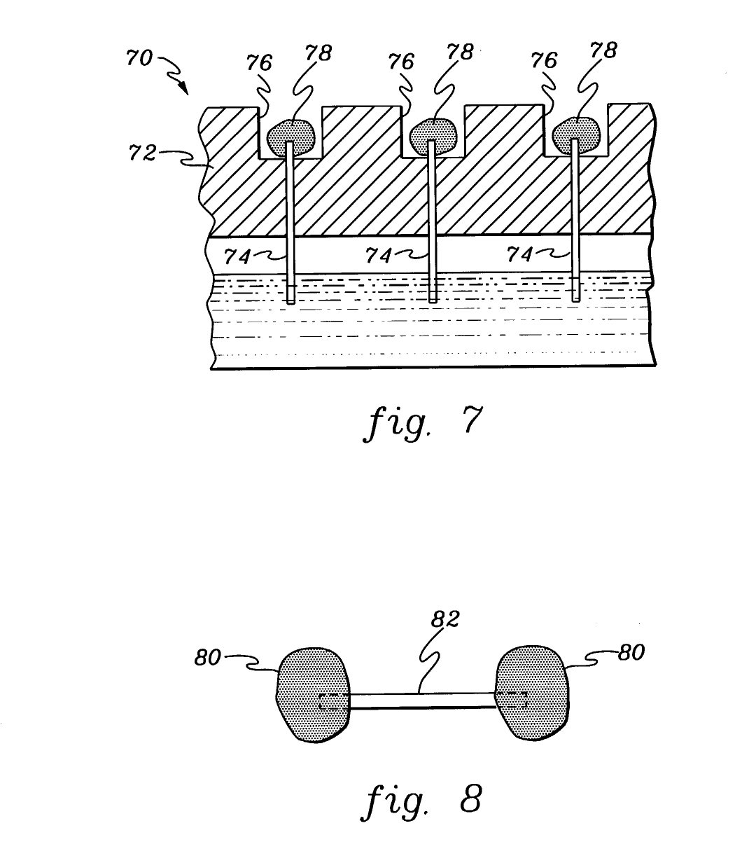

FIG. 7 illustrates another fluid delivery device comprising a template or support 72 having a plurality of nanotubes 74. Each of the nanotubes extends into one of a plurality of cutouts 76 in support 72. One end of the nanotube pierces the cell 78 and the other end of the nanotubes is disposed in a fluid. The fluid can be delivered under pressure to the cell or via capillary action. Such a device allows forming an array for testing a plurality of cell at the same time.

Whether a cell is supported on a substrate or using a fluid delivery device, a method for delivering a fluid, e.g., a drug or a chemical, into a cell may include inserting a nanotube into the cell and delivering the fluid into the cell via the nanotube. In addition, the use of nanotubes allows delivering the fluid to a specific location of a cell and without damaging the cell.

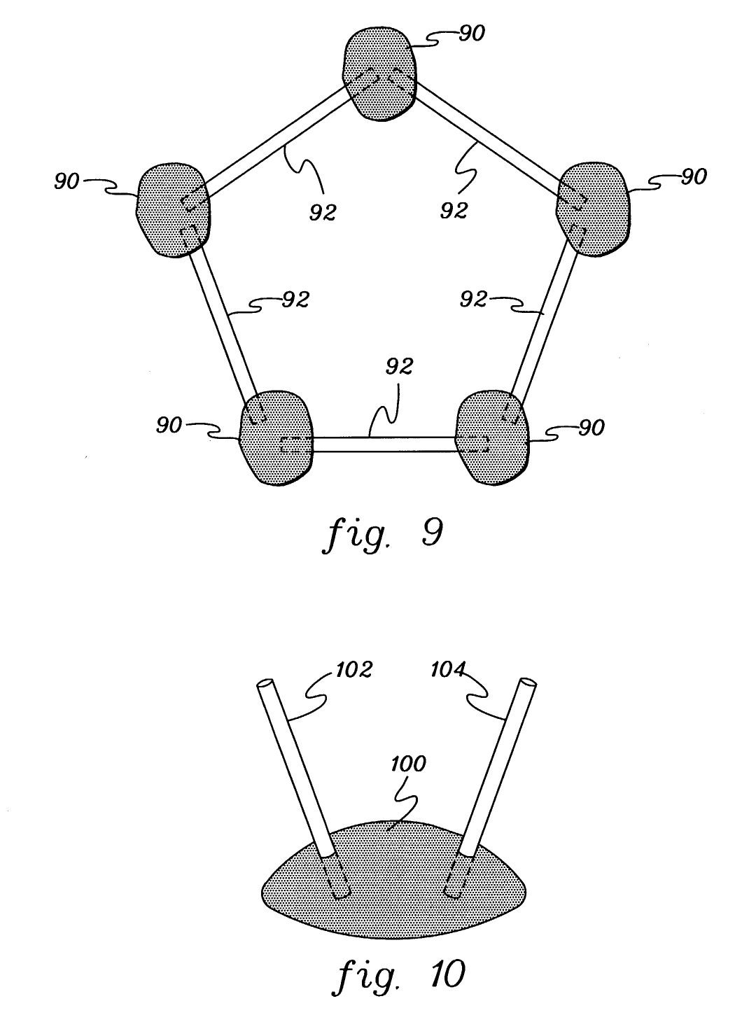

FIG. 8 illustrates two spaced-apart cells 80 connected by at least one nanotube 82. Each end of the nanotube pierces one of the cells. FIG. 9 illustrates a plurality of cells 90 connected by a plurality of nanotubes 92. The method for fluidly connecting two or more cells without damaging the cells may include inserting one end of a nanotube into a first cell and inserting the other end of the nanotube into a second cell, and exchanging biological matter between the cells via the nanotube. As noted above, using nanotubes allows transferring fluid between cells and without damaging the cells.

FIG. 10 illustrates a cell 100 pierced by two spaced-apart nanotubes 102 and 104. As described above, the nanotube may be a single-wall nanotube, a multi-wall nanotube, a nanotube bundle, and combinations thereof. The arrangement shown in FIG. 10, as well as the arrangement shown in FIG. 3, allow sensing electrical properties of the cell. For example, the two nanotubes shown in FIG. 10 or the nanotube and the contact shown in FIG. 3 may be used as electrodes for detecting changes in electrical potential therebetween. The two nanotubes shown in FIG. 10, or the nanotube and contact shown in FIG. 3 may also be used to apply an electrical potential to the cells.

For example, with reference to FIG. 10, a method for sensing a property of a cell may include inserting two nanotubes into the cell, measuring an electrical potential or resistance between the two nanotubes, and relating the electrical potential or resistance to a property of the cell (e.g., the cell is dead or alive, cancerous or benign). In addition, a method for sensing a property of a cell may include inserting two nanotubes into the cell, applying an electrical potential (voltage such as with a given frequency or amplitude) to the two nanotubes, and sensing a property of the cell (e.g., chemical changes, survival of the cell, etc.). Still another method for sensing a property of a cell may include inserting two nanotubes into the cell, applying a plurality of electrical potentials (e.g. varying voltages such as a varying frequency or varying amplitude) to the two nanotubes, sensing a response spectrum (e.g., chemical changes) of the cell based on the applied plurality of electrical potentials, and correlating the response spectrum to a property or characteristic of the cell (e.g., to identify or distinguish a sick cell, an old cell, a new cell, the type of cell, etc.).

With reference again to FIG. 3, a method for sensing a property of a cell may include supporting a cell on a contact, inserting a nanotube into the cell, measuring an electrical potential or resistance between the contact and the nanotube, and relating the electrical potential or resistance to a property of the cell. A method for sensing a property of a cell may also include supporting a cell on a contact, inserting a nanotube into the cell, applying an electrical potential (voltage such as with a given frequency or amplitude) to the contact and the nanotube, and sensing a property of the cell (e.g., chemical changes, survival of the cell, etc.). Still another method for sensing a property of a cell may include supporting a cell on a contact, inserting a nanotube in the cell, applying a plurality of electrical potentials (e.g. varying voltages such as a varying frequency or varying amplitude) to the nanotube and the contact, sensing a response spectrum (e.g., chemical changes) of the cell based on the applied plurality of electrical potentials, and correlating the response spectrum to a property or characteristic of the cell (e.g., to identify or distinguish a sick cell, an old cell, a new cell, the type of cell, etc.).

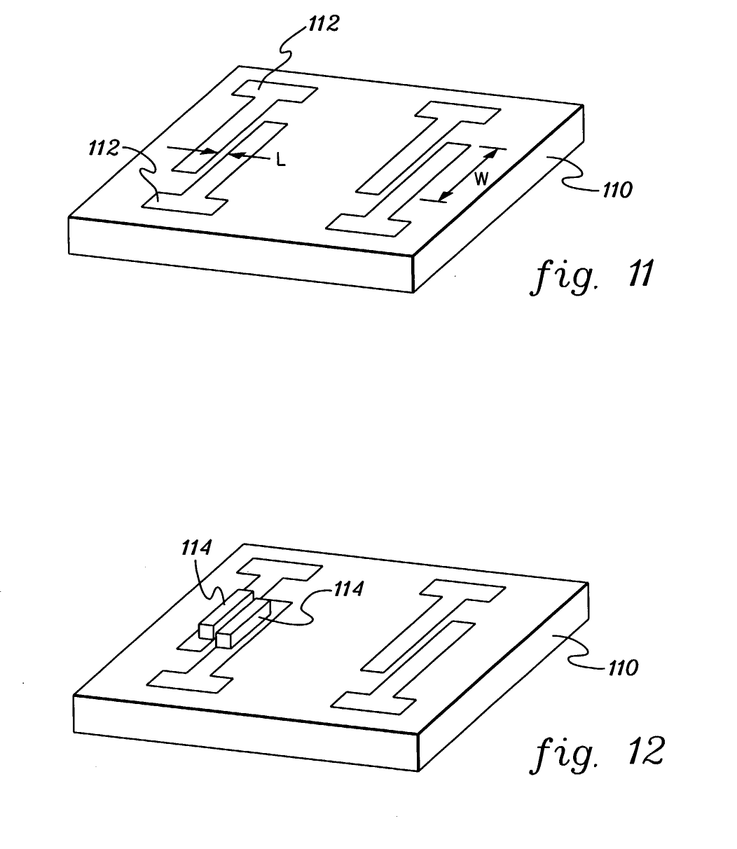

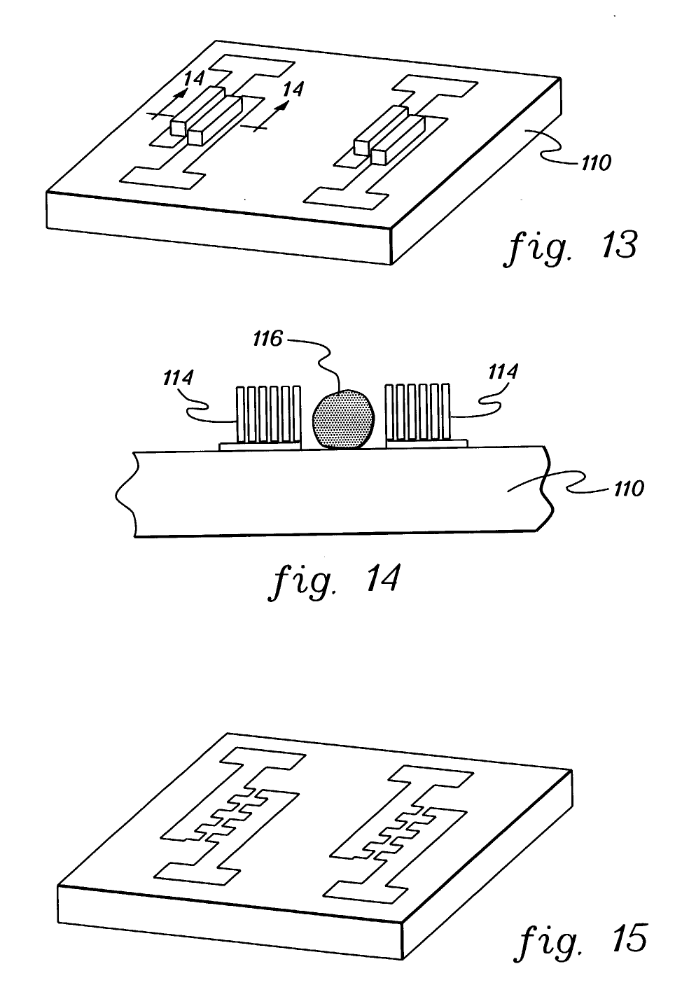

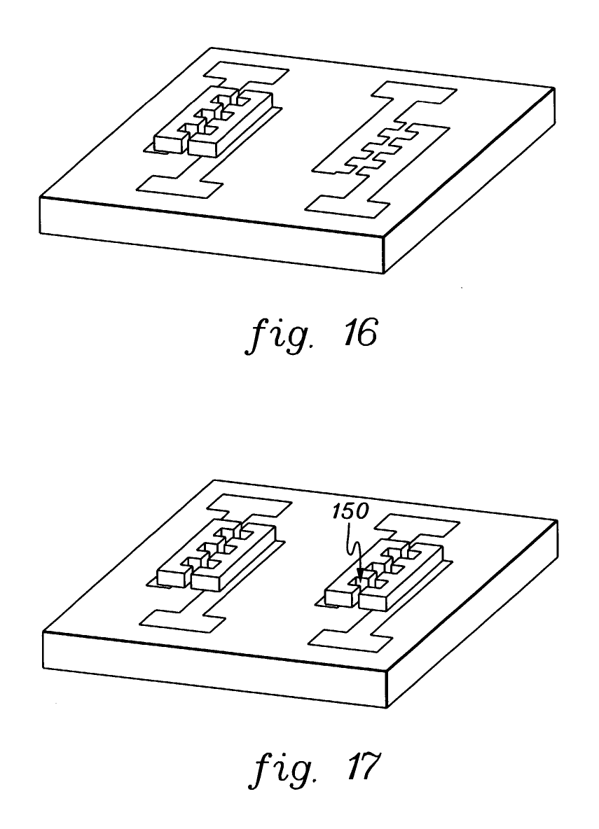

FIGS. 11-14 illustrate the steps for fabricating a test setup for parallel cell impedance spectra sensing. The nanotubes 114 may be patterned on an insulating substrate 110 and built into vertically aligned arrays or bundles. In this example, the fabrication starts with insulating substrate 110 and depositing spaced-apart metal plates 112. Insulating GaN, for example, may be deposited to selectively prevent the deposition of carbon nanotubes on the metal plates. A width W of the adjacent metal plates may vary from about few microns to hundreds of microns. For example, the width may be about 50 microns. The pad dimensions may be large enough for bonding, e.g., about 150 microns by about 150 microns. A gap L between the metal plates may vary from deep submicron dimensions to a few microns, and the gap may be about 3 microns. FIGS. 15-17 illustrate the steps for fabricating another test setup for individual cell impedance spectra sensing. In this setup, a plurality of recesses or cavities 150 (FIG. 17) are formed between the nanotubes for receiving a cell therein. The cavities may be sized at less than about 3 microns by less than about 3 microns.

Multi-wall nanotube arrays may also be grown on silica patterns. One recent method relies on chemical vapor deposition of hydrocarbons (e.g. xylene) and subsequent catalyst delivery. The nanotube arrays are selectively grown on patterned silica islands on Si substrate. The arrays consist of many nanotubes, aligned parallel to each other.

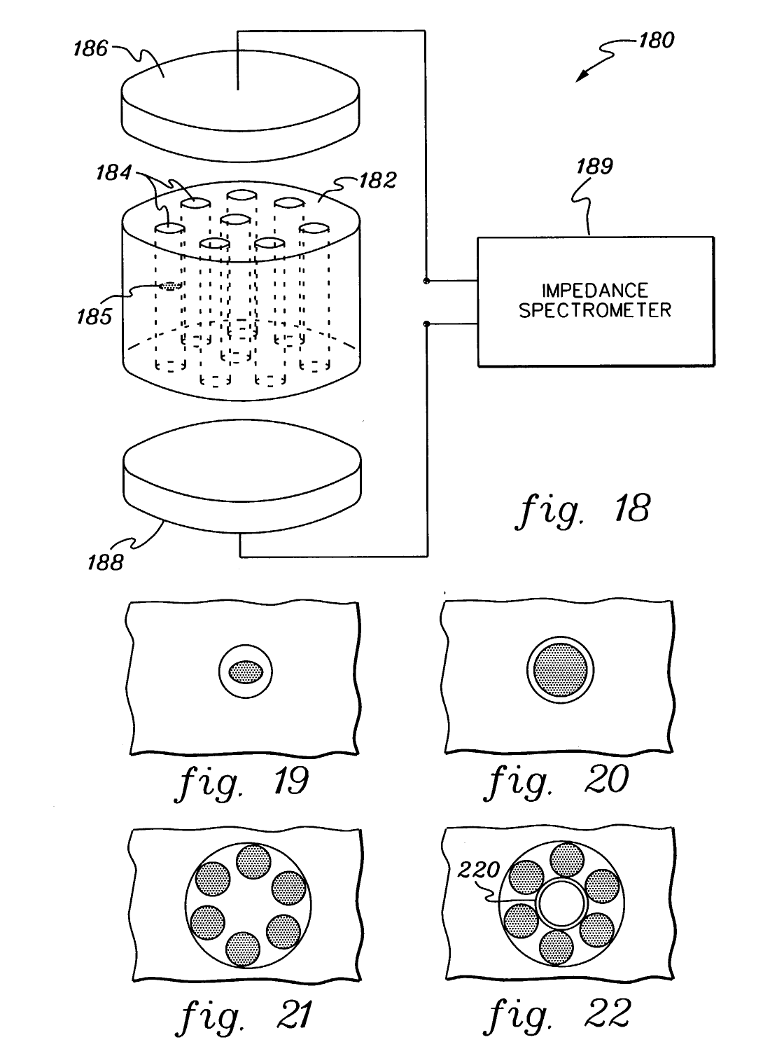

FIG. 18 illustrates a test setup 180 for cell impedance spectroscopy of cells which includes a porous alumina template or support 182 having a plurality of passageways 184 therethrough and a top contact 186 and bottom contact 188 disposed above and below the support. The contacts may be connected to an impedance spectrometer 189. One or more cells 185 may be placed in support 182 in various possible arrangements. FIGS. 19-22 illustrate four possible cell arrangements inside the passageway of the support. For example, FIGS. 19 and 20 illustrate a single cell contained in the passageways of the support while FIGS. 21 and 22 illustrate a plurality of cells disposed in the support. In addition, in FIG. 22, a nanotube 220 may be grown inside the passageway in the support and the cells may be disposed between the nanotube and the surface of the passageway of the support.

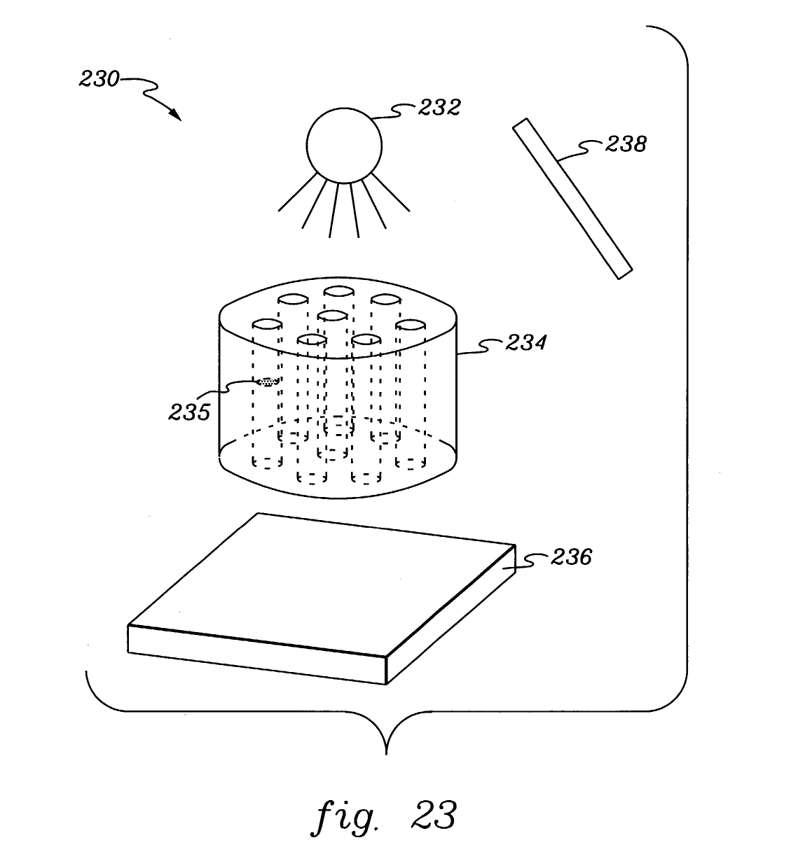

FIG. 23 illustrates a test setup 230 for cell spectroscopy in accordance with the present invention. A light or electromagnetic radiation source 232 may be placed above a support 234 having a plurality of passageways and a spectrometer or a detector 236 may be placed below the support for studying cell absorption and photoluminescence spectra. One or more cells 235 may be placed in passageways of the support in many possible arrangements, for example, as described above. In addition, a spectrometer or a detector 238 may be disposed above the support for detecting reflective emissions from the one or more cells.

Further embodiments of the present invention may include a combination fluid delivery device and cell sensing device by combining the various devices described above. For example, a cell fluid delivery device and cell sensing device may include a plurality of nanotubes piercing the cell with two nanotubes used as electrodes for sensing electric properties of the cell and other nanotubes used as either a gas or a liquid delivery channel. A method for delivering a fluid and sensing a property of a cell may include introducing a fluid through a nanotube inserted into the cell, and detecting an electrical potential between two nanotubes inserted into the cell or detecting an electrical potential between the nanotube and a substrate on which the cell is supported. Some of the nanotubes piercing the cell may be used as probes for sensing the state of the cell upon administration of one or more biologically active agents for use in drug discovery. Cell piercing nanotubes may also be used as a gas or a liquid delivery device and as chemical or genetic probes.

Thus, while various embodiments of the present invention have been illustrated and described, it will be appreciated to those skilled in the art that many changes and modifications may be made thereunto without departing from the spirit and scope of the invention.

1. A method for delivering a fluid into a cell, the method comprising:
inserting a first end of a nanotube into the cell;
connecting a second end of the nanotube to a fluid supply; and
transferring fluid from the fluid supply into the cell via the nanotube.

2. The method of claim 1 further comprising attaching the cell to a support.

3. The method of claim 1 further comprising forming a nanotube in a passageway of a support, and removing a portion of the support to expose the first end of the nanotube.

4. The method of claim 1 further comprising forming a nanotube in a passageway of a support, and forming a cutout in the support into which the first end of the nanotube extends.

5. The method of claim 1 wherein the fluid comprises at least one of a drug and a chemical.

6. A fluid delivery device for delivering a fluid to at least one cell, said fluid delivery device comprising:
a support having a passageway therethrough;
a nanotube disposed in said passageway, said nanotube having a first end extending from a surface of said support; and
a sidewall extending from said support to form a container for containing a fluid in fluid communication with a second end of said nanotube.

7. The fluid delivery device of claim 6 wherein said passageway comprises a plurality of spaced-apart passageways, said at least one nanotube comprises a plurality of nanotubes each of which being disposed in a different one of said plurality of passageways, and said plurality of nanotubes having a plurality of first ends extending from said surface of said support.

8. A fluid delivery device for delivering a fluid to at least one cell, the fluid delivery device comprising:
a support having a passageway therethrough which opens into a cutout on a surface of said support;
at least one nanotube disposed in said at least one passageway and having a first end of the nanotube extending into said cutout; and
said cutout being configured for receiving at least one cell into which said first end of the nanotube is insertable.

9. The fluid delivery device of claim 8 further comprising a container in fluid communication with a second end of the nanotube.

10. The fluid delivery device of claim 9 wherein the passageway comprises a plurality of spaced-apart passageways, and the at least one nanotube comprises a plurality of nanotubes each of which being disposed in a different one of the plurality of passageways and extending into a different one of a plurality of cutouts extending around openings of the plurality of passageways.

11. A method for forming a fluid delivery device, the method comprising:
providing a support having a passageway therein;
forming a nanotube in the passageway; and
removing a portion of the support to expose a first end of the nanotube.

12. The method of claim 11 further comprising attaching a sidewall to the support to form a container for containing fluid in fluid communication with a second end of the nanotube.

13. The method of claim 11 wherein the removing comprises forming a cutout in the support around the exposed first end of the nanotube for receiving a cell in the cutout.

14. The method of claim 13 further comprising attaching a sidewall to the support to form a container for containing fluid in fluid communication with a second end of the nanotube.

15. A method for fluidly connecting two or more cells, the method comprising:
inserting one end of a nanotube into a first cell; and
inserting the other end of the nanotube into a second cell.

16. The method of claim 15 further comprising exchanging biological matter between the cells via the nanotube.

17. A method for determining a property of a cell, the method comprising:
inserting two nanotubes into the cell;
measuring at least one of a voltage and a resistance between the two nanotubes; and
relating the at least one of the voltage and the resistance to a property of the cell.

18. A method for sensing a property of a cell, the method comprising:
inserting two nanotubes into the cell;
applying a voltage to the two nanotubes; and
sensing a property of the cell.

19. The method of claim 18 wherein the applying comprises applying a voltage having at least one of a generally constant frequency and a generally constant amplitude.

20. The method of claim 18 wherein the applying comprises applying a varying voltage, and the sensing comprises sensing a response spectrum of the cell based on the varying voltage and relating the response spectrum to the property of the cell.

21. The method of claim 20 wherein the varying voltage comprises at least one of the varying voltage having a varying frequency and the varying voltage having a varying amplitude.

22. A method for determining a property of a cell, the method comprising:
supporting the cell on a contact;
inserting a nanotube into the cell;
measuring at least one of a voltage and a resistance between the contact and the nanotube; and
relating the at least one of the voltage and the resistance to a property of the cell.

23. A method for sensing a property of a cell, the method comprising:
supporting the cell on a contact;
inserting a nanotube into the cell;
applying a voltage to the contact and the nanotube; and
sensing a property of the cell.

24. The method of claim 23 wherein the applying comprises applying a voltage having at least one of a generally constant frequency and a generally constant amplitude.

25. The method of claim 23 wherein the applying comprises applying a varying voltage, and the sensing comprises sensing a response spectrum of the cell based on the varying voltage and relating the response spectrum to the property of the cell.

26. The method of claim 25 wherein the varying voltage comprises at least one of the varying voltage having a varying frequency and the varying voltage having a varying amplitude.

27. An apparatus for use in measuring the impedance spectra of a cell, said apparatus comprising:
a nonconductive support;
two spaced-apart conductive pads;
a first and second plurality of nanotubes each of which extending from a respective one of said two conductive pads; and
wherein at least one of said first and said second plurality of nanotubes being spaced apart a distance for receiving a cell therein, and the first and second nanotubes defining a plurality of cavities for receiving individual cells therein.

28. The apparatus of claim 27 wherein the first and second plurality of nanotubes are spaced apart a distance less that about 3 microns.

29. The apparatus of claim 27 wherein the cavities are sized at less than about 3 microns by less than about 3 microns.

30. A method for measuring an impedance spectra of at least one cell, the method comprising:
providing a nonconductive support;
depositing two spaced-apart conductive pads on the support;
forming a first and a second plurality of nanotubes extending from the conductive pads wherein at least one of the first and the second nanotubes being spaced-apart a distance for receiving a cell therein and the first and the second nanotubes defining a plurality of cavities for receiving individual cells therein; and
measuring the impedance spectra using an impedance spectrometer attached to the conductive pads.

31. An apparatus for measuring an impedance spectra of at least one cell, said apparatus comprising;
a nonconductive support having at least one passageway for receiving the at least one cell; and
a pair of contacts disposed on opposite ends of the support and connectable to an impedance spectrometer.

32. An apparatus for measuring a radiation spectra of at least one cell, said apparatus comprising:
a support having at least one passageway for receiving the at least one cell;
a radiation source disposed adjacent the support; and
a detector disposed adjacent said support for detecting radiation from said at least one cell.

33. The apparatus of claims 32 wherein the radiation source is disposed on one side of the at least one cell, and the detector is disposed on an opposite side of the at least one cell.

34. The apparatus of claims 32 wherein the radiation source and the detector are disposed on the same side of the at least one cell.

35. A method of delivering a fluid and sensing a property of a cell, the method comprising:
inserting a nanotube into the cell;
introducing a fluid through the nanotube into the cell;
detecting a property of the cell using the nanotube.

36. The method of claim 35 wherein the inserting comprises inserting a plurality of nanotubes into the cell, the introducing comprises introducing the fluid through one of the plurality of nanotubes, and the detecting comprises detecting an electrical potential between at least two of the plurality of nanotubes.

37. The method of claim 35 wherein the detecting comprises detecting an electrical potential between the nanotube and a support on which the cell is attached.
